(19) United States
(12) Patent Application Publication
(10) Pub. No.: US 2009/0324761 A1
(43) Pub. Date: Dec. 31, 2009

(54) Methods And Compositions for Improving Gastrointestinal Health

(75) Inventors: Christina Khoo, Pembroke, MA (US); Fernando Qvyjt, Topeka, KS (US)

Correspondence Address: COLGATE-PALMOLIVE COMPANY, 909 RIVER ROAD, PISCATAWAY, NJ, 08855, US

(73) Assignee: Hill's Pet Nutrition, Inc., Topeka, KS (US)

(21) Appl. No.: 12/090,945

(22) PCT Filed: Oct. 19, 2006
(86) PCT No.: PCT/US06/60064

§ 371 Date: Dec. 19, 2008

Related U.S. Application Data
(60) Provisional application No. 60/728,151, filed on Oct. 19, 2005.

(51) Int. Cl. A61K 36/00; A61P 1/00

(52) U.S. Cl. 424/777

(57) ABSTRACT

Methods for maintaining or improving the gastrointestinal health of an animal by administering a gastrointestinal health maintaining or improving amount of at least one banana musa fruit to the animal susceptible to or suffering from poor gastrointestinal health. In one embodiment, banana is administered to the animal to prevent or treat diarrhea or to improve stool quality. In other embodiments, the banana musa fruit is administered in conjunction with one or more of foods, prebiotics, probiotics, and anti-diarrhea drugs.

### CROSS-REFERENCE TO RELATED APPLICATIONS

This application claims priority to U.S. Provisional Application Ser. No. 60/728,151 filed Oct. 19, 2005, the disclosure of which is incorporated herein by reference.

### BACKGROUND OF THE INVENTION

1. Field of the Invention

The invention relates generally to methods and compositions for maintaining and improving gastrointestinal health and particularly to using banana musa fruits for maintaining and improving gastrointestinal health.

2. Description of the Related Art

Banana musa fruits, particularly the banana and plantain family, have provided man with food, clothing, tools, and shelter since the beginning of recorded history. Bananas and plantains are today grown in every humid tropical region and constitute the 4th largest fruit crop of the world. World banana consumption is expected to be about 15 million tons in 2010. Bananas contain about 74% water, 23% carbohydrates, 1% proteins, 0.5% fat, and 2.6% fiber, depending on the banana cultivar, degree of ripeness, and growing conditions. Bananas are known to be a good source of energy and a rich source of potassium.

US Patent Application No. 20010022986 discloses using banana flavorants in electrolyte beverages useful as a sports drink or as a therapeutic to replace electrolyte loss. U.S. Pat. No. 4,597,970 discloses the use of banana oil as a flavorant in chewing gum. U.S. Pat. No. 6,093,425 discloses banana flavored milk compositions.

Animals need good gastrointestinal health for their ordinary well being but, unfortunately, poor gastrointestinal health is common. Poor gastrointestinal health can be quite serious and require medical attention, e.g., Crohn's disease; irritable bowel syndrome, and other such chronic conditions. Other forms of poor gastrointestinal health are less serious and can be essentially self-limiting, e.g., food borne viruses and intestinal flu that often result in diarrhea, poor stool quality, or other symptoms of poor gastrointestinal health. Poor gastrointestinal health results from various causes. For example, small intestinal bacterial overgrowth (IBO) occurs in people and companion animals such as dogs, cats, and horses. IBO may be caused by poor motility, food retention, or decreased gastric acidity. Further, animals must efficiently and properly digest food to maintain good health. However, poor gastrointestinal health interferes with the ordinary digestion of food and adversely affects an animal's health and well being.

Many gastrointestinal disorders are accompanied by diarrhea, a loose watery stool that can be extremely unpleasant to an animal or its caregiver. Further, some foods tend to create or exacerbate diarrhea. For example, U.S. Pat. No. 6,280,779 discloses that diarrhea can be a significant problem with pets consuming a “chunks and gravy” diet. Similarly, poor stool quality is often indicative of milder forms of poor gastrointestinal health that do not result in diarrhea.

Current methods for maintaining and improving gastrointestinal health often involve modifying the diet, administering various food ingredients thought to effect gastrointestinal health, or administering various drugs thought to be useful for maintaining or improving gastrointestinal health. These methods, while useful, have not solved the problem. There is, therefore, a need for new methods and compositions for maintaining and improving gastrointestinal health in an animal.

### SUMMARY OF THE INVENTION

It is, therefore, an object of the present invention to provide methods and compositions useful for maintaining or improving gastrointestinal health.

It is another object of the invention to provide methods and compositions useful for preventing or treating diarrhea.

It is another object of the invention to provide methods and compositions useful for improving stool quality.

It is a further object of the invention to provide articles of manufacture in the form of kits that contain combinations of compositions and other components useful for maintaining or improving gastrointestinal health.

These and other objects are achieved using novel methods and compositions for improving gastrointestinal health in an animal. The methods comprise administering a gastrointestinal health improving amount of at least one banana musa fruit to an animal susceptible to or suffering from poor gastrointestinal health. The compositions comprise at least one banana musa fruit and at least one ingredient selected from the group consisting of (1) an ingredient suitable for consumption by an animal, (2) at least one of a gastrointestinal health improving agent selected from the group consisting of probiotics, prebiotics, and anti-diarrhea drugs, and (3) at least one microbial exopolysaccharide in amounts sufficient to maintain or improve gastrointestinal health.

Banana musa fruits and the compositions of the present invention are surprisingly effective for maintaining or improving gastrointestinal health, particularly for preventing or treating diarrhea and for improving stool quality, and therefore can be used to improve the health and wellness of the animal.

Other and further objects, features, and advantages of the present invention will be readily apparent to those skilled in the art.

### DETAILED DESCRIPTION OF THE INVENTION

### Definitions

The term “animal” means any animal susceptible to or suffering from poor gastrointestinal health. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

The term “in conjunction” means that one or more of the compositions or compounds (e.g., anti-diarrhea drugs or composition components) of the present invention are administered to an animal (1) together in a food composition or (2) separately at the same or different frequency using the same or different administration routes at about the same time or periodically. “Periodically” means that the compositions, food compositions, and compounds are administered on a dosage schedule acceptable for a specific composition, food composition, and compound and that the food compositions are administered or fed to an animal routinely as appropriate for the particular animal. “About the same time” generally means that the compositions, composition components, anti-diarrhea drugs, and food compositions are administered at the same time or within about 72 hours of each other. In conjunction specifically includes administration schemes wherein anti-diarrhea drugs are administered for a prescribed period and the compositions are administered indefinitely.

The term “single package” means that the components of a kit are physically associated in or with one or more containers and considered a unit for manufacture, distribution, sale, or use. Containers include, but are not limited to, bags, boxes, bottles, shrink wrap packages, stapled or otherwise affixed components, or combinations thereof. A single package may be containers of individual food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag containing one component and directions instructing the user to go to a website, contact a recorded message, view a visual message, or contact a caregiver or instructor to obtain instructions on how to use the kit.

The term “anti-diarrhea drug” means any compound, composition, or drug useful for preventing or treating diarrhea.

The term “banana musa fruit” means the fruit in any form suitable for administration to an animal, including raw fruits, cooked fruits, fruit extracts, fruit chips, and the like.

### THE INVENTION

In one aspect, the present invention provides methods for maintaining or improving the gastrointestinal health of an animal. The methods comprise administering a gastrointestinal health maintaining or improving amount of at least one banana musa fruit to an animal susceptible to or suffering from poor gastrointestinal health. In one embodiment, the methods comprise maintaining or improving gastrointestinal health by preventing or treating diarrhea. In another, the methods comprise improving gastrointestinal health by improving stool quality.

The banana musa fruit useful in the present invention is any banana musa fruit capable of maintaining or improving gastrointestinal health. Examples of suitable banana musa fruits for use in the present invention include plantains, bananas, and combinations thereof. Preferably, the banana musa fruit is a banana.

Generally, banana musa fruits are administered to an animal in amounts of from about 0.02 to about 3 g/kg body weight per day, preferably from 0.2 to about 1.5 g/kg body weight per day.

The banana musa fruit is administered to the animal using any suitable means, e.g., orally. The fruit is administered orally using any suitable form for oral administration, e.g., tablets, pills, suspensions, solutions, emulsions, capsules, powders, syrups, and palatable feed compositions such as a confectionery for a human or a treat or flavored treat for a domesticated animal.

In a further aspect, the methods of the present invention further comprise administering the banana musa fruit in conjunction with one or more ingredients suitable for consumption by an animal. In a preferred embodiment, the banana musa fruit and ingredients are admixed prior to administration to the animal. Generally, the banana musa fruit comprises from about 0.1% to about 5% of the mixture, preferably from about 0.2% to about 3% and most preferably from about 0.3% to about 1.5% by weight of the composition. The banana musa fruit should be administered in amounts that are not toxic or otherwise deleterious to an animal's health. Unless otherwise specifically indicated, all weights for the compositions of the present invention are based on dry weight of a composition after all components and ingredients are admixed.

Any ingredient suitable for consumption by an animal can be used in the present invention. In one embodiment, the ingredient is a food ingredient known to skilled artisans, preferably ingredients known to promote the health of an animal. Typical food ingredients include, but are not limited to, fats, carbohydrates, proteins, fibers, and nutritional agents such as vitamins, minerals, and trace elements. Skilled artisans can select the amount and type food ingredients based upon the dietary requirements of the animal, e.g., the animal's species, age, size, weight, health, and function.

In another aspect, the methods of the present invention further comprise administering the banana musa fruit in conjunction with at least one of a gastrointestinal health improving agent selected from the group consisting of probiotics, prebiotics, and anti-diarrhea drugs.

Probiotics useful in the present invention are live microorganisms that have a beneficial effect in the prevention and treatment of specific medical conditions when ingested. Probiotics are believed to exert biological effects through a phenomenon known as colonization resistance. The probiotics facilitate a process whereby the indigenous anaerobic flora limits the concentration of potentially harmful (mostly aerobic) bacteria in the digestive tract. Other modes of action, such as supplying enzymes or influencing enzyme activity in the gastrointestinal tract, may also account for some of the other functions that have been attributed to probiotics. Prebiotics are nondigestible food ingredients that beneficially affect host health by selectively stimulating the growth and/or activity of bacteria in the colon. The prebiotic, fructooligosaccharide (FOS) is found naturally in many foods such as wheat, onions, bananas, honey, garlic, and leeks. FOS can also be isolated from chicory root or synthesized enzymatically from sucrose. FOS fermentation in the colon results in a large number of physiologic effects including increasing the numbers of bifidobacteria in the colon, increasing calcium absorption, increasing fecal weight, shortening of gastrointestinal transit time, and possibly lowering blood lipid levels. The increase in bifidobacteria has been assumed to benefit human health by producing compounds to inhibit potential pathogens, by reducing blood ammonia levels, and by producing vitamins and digestive enzymes. Probiotic bacteria such as Lactobacilli or Bifidobacteria are believed to positively affect the immune response by improving the intestinal microbial balance leading to enhanced antibody production and phagocytic (devouring or killing) activity of white blood cells. Bifidobacterium lactis could be an effective probiotic dietary supplement for enhancing some aspects of cellular immunity in the elderly. Probiotics enhance systemic cellular immune responses and may be useful as a dietary supplement to boost natural immunity in otherwise healthy adults. Probiotics include many types of bacteria but generally are selected from four genera of bacteria: Lactobacilllus acidophilus, Bifidobacteria, Lactococcus, and Pediococcus. The amount of probiotics and prebiotics to be administered to the animal is determined by the skilled artisan based upon the type and nature of the probiotic and prebiotic and the type and nature of the animal, e.g., the age, weight, general health, sex, extent of microbial depletion, presence of harmful bacteria, and diet of the animal. Generally, probiotics are administered to the animal in amounts of from about one to about twenty billion colony forming units (CFUs) per day for the healthy maintenance of intestinal microflora, preferably from about 5 billion to about 10 billion live bacteria per day. Generally, prebiotics are administered in amounts sufficient to positively stimulate the healthy microflora in the gut and cause these “good” bacteria to reproduce. Typical amounts are from about one to about 10 grams per serving or from about 5 percent to about 40 percent of the recommended daily dietary fiber for an animal. The probiotics and prebiotics can be made part of the composition by any suitable means. Generally, the agents are mixed with the composition or applied to the surface of the composition, e.g., by sprinkling. When the agents are part of a kit, the agents can be admixed with other materials or in their own package.

Anti-diarrhea drugs useful in the invention are any anti-diarrhea drugs known to skilled artisans to be useful for combating diarrhea, e.g., loperamide, diphenoxylate, pancrelipase, and tincture of opium. Holistic anti-diarrhea drugs and compositions are also included in the present invention, e.g., peppermint and ginger. The anti-diarrhea drugs are administered to the animal using any method appropriate for the anti-diarrhea drug and in amounts known to skilled artisans to be sufficient to treat or prevent diarrhea. The anti-diarrhea drugs have a beneficial effect on the gastrointestinal tract by reducing the incidence or severity of diarrhea.

In a further aspect, the method of the present invention further comprises administering the banana musa fruit in conjunction with at least one microbial exopolysaccharide.

The microbial exopolysaccharides useful in the present invention is any microbial exopolysaccharide capable of preventing or treating diarrhea. Examples of suitable microbial exopolysaccharides for use in the present invention generally include those microbial exopolysaccharides synthesized from bacteria selected from the group consisting of Sphingomonas paucimobilis, Agrobacterium biovar, Xanthomonas campestris, Alcaligenes species, Aureobasidium pullulans, Acetobacter xylinum, Sclerotium rolfsii, Schizophyllum commune, Saccharomyces cerevisiae and Sclerotium glucanicum. In some embodiments, the microbial exopolysaccharide is selected from the group consisting of rhamsan, curdlan, xanthan gum, scleroglucan, PS-10 gum, PS-21 gum, PS-53 gum, polysaccharides from Alcaligenes species, PS-7 gum, gellan gum, curdlan, bacterial alginate, dextran, pullulan, baker's yeast glycan, bacterial cellulose, 6-deoxy-hexose-containing polysaccharides, and combinations thereof.

In a preferred embodiment, the microbial exopolysaccharide comprises a gellan gum. Gellan gum is a linear polysaccharide made from fermentation by Sphingomonas paucimobilis (elodea) (ATCC31461). Industrial preparation of the gum can be carried out by inoculating Sphingomonas paucimobilis into a fermentation broth containing glucose, glucuronic acid and rhamnose to form a tetrasaccharide repeating unit in a ratio of 2:1:1. In its native form, gellan gum is highly acylated with 1.5 acyl group, acetyl and glycerate, per repeating unit. Modifications of the acyl groups both in number and type can be made as long as the basic anti diarrhea activity of the gellan gum is not significantly diminished. These different forms can be obtained from CP Kelco under different tradenames including Gelrite®, K9A50 and other Kelco gellan gums including but not limited to, Kelcogel LT®, Kelcogel F, and Kelcogel LT100®. As used throughout the specifications “gellan” refers to the natural gum or acyl modified gum as long as the anti-diarrhea function is maintained.

Generally, the microbial exopolysaccharide is administered in amounts of from about 0.05 to about 2 g/kg body weight per day.

The banana musa fruit can be administered in conjunction in various combinations of food ingredients, probiotics, prebiotics, anti-diarrhea drugs, and microbial exopolysaccharides. For example, the banana musa fruit can be administered in conjunction with a prebiotic to maintain gastrointestinal health. Similarly, the banana musa fruit can be administered in conjunction with a probiotic and anti-diarrhea drug to treat diarrhea and improve gastrointestinal health.

In another aspect, the invention provides compositions useful for maintaining or improving the gastrointestinal health of an animal. The compositions comprise a gastrointestinal health maintaining or improving amount of at least one banana musa fruit and at least one ingredient selected from the group consisting of (1) an ingredient suitable for consumption by an animal, (2) at least one of a gastrointestinal health improving agent selected from the group consisting of probiotics, prebiotics, and anti-diarrhea drugs, and (3) at least one microbial exopolysaccharide.

The present invention provides a variety of compositions comprising banana musa fruits, e.g., foods, supplements, treats, and toys. In one embodiment, the composition comprises a food for animal consumption comprising one or more food ingredients and one or more banana musa fruits. In various embodiments, the food ingredient is selected from the group consisting of meat, meat by-products, a dairy products, egg products, and combinations thereof. Generally, the food component is sufficient to meet the nutritional needs of the animal, e.g., a cat or a dog. Although both liquid and solid foods are provided, solid foods are typically preferred. If the food is solid, the banana musa fruit may be coated on the food, incorporated into the food, or both. In another embodiment, the composition comprises an animal feed supplement. Supplements include, for example, a feed used with another feed to improve the nutritive balance or performance of the total. Possible supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Inc. Official Publication, page 220 (2003). Supplements may be in various forms including, for example, powders, liquids, syrups, pills, encapsulated compositions, and the like. In a further embodiment, the composition comprises an animal treat. Treats include, for example, compositions that are given to an animal to entice the animal to eat during a non-meal time. Contemplated treats for canines include, for example, dog bones. Treats may be nutritional, wherein the composition comprises one or more nutrients and may, for example, have a composition as described above for food. Non-nutritional treats encompass any other treats that are non-toxic. The banana musa fruit can be coated onto the treat, incorporated into the treat, or both. In another embodiment, the composition comprises a toy. Toys include chewable and consumable toys. Possible toys for dogs include artificial bones. The banana musa fruit forms a coating on the surface of the toy or on the surface of a component of the toy, are incorporated partially or fully throughout the toy, or both. In an embodiment, the banana musa fruit is orally accessible by the intended user. There a wide range of suitable toys currently marketed. See, e.g., U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). The invention encompasses both partially consumable toys (e.g., toys comprising plastic components) and fully consumable toys (e.g., rawhides and various artificial bones). The invention also encompasses toys for both human and non-human use, particularly for companion, farm, and zoo animal use, and particularly for dog or cat use.

The administration of banana musa fruit to an animal is useful for maintaining or improving the gastrointestinal health of an animal, particularly for preventing or treating diarrhea and for improving stool quality. Specifically, the administration of banana musa fruit to an animal has been shown to prevent diarrhea in an animal that has a tendency to have diarrhea from time to time and to reduce diarrhea in an animal already experiencing diarrhea. Similarly, the administration of banana musa fruit to an animal has been shown to improve stool quality in an animal that has poor gastrointestinal health that has not progressed to diarrhea.

The methods and compositions of the invention are useful for a variety of human and non-human animals susceptible to or suffering from poor gastrointestinal health, including avian, bovine, canine, equine, feline, hicrine, murine, ovine, and porcine animals. In some embodiments, the animal is a companion animal such as canine or feline, particularly a dog or a cat.

In a further aspect, the present invention provides kits suitable for administering banana musa fruits to an animal. The kits comprise in separate containers in a single package or in separate containers in a virtual package, as appropriate for the kit component, at least one banana musa fruit and at least one of (1) a different banana musa fruit, (2) one or more ingredients suitable for consumption by an animal, (3) one or more microbial exopolysaccharides, (4) one or more gastrointestinal tract improving agents selected from the group consisting of probiotics and prebiotics, (5) one or more anti-diarrhea drugs, (6) instructions for how to combine the banana musa fruit and other kit components to maintain or improve gastrointestinal health, particularly to produce a composition useful for preventing or treating diarrhea or for improving stool quality, and (7) instructions for how to use the banana musa fruits and other components of the present invention, particularly for the benefit of the animal. When the kit comprises a virtual package, the kit is limited to instructions in a virtual environment in combination with one or more physical kit components. The kit contains the banana musa fruit and other components in amounts sufficient to maintain or improve gastrointestinal health. Typically, the banana musa fruits and the other suitable kit components are admixed just prior to consumption by an animal. In one embodiment, the kit contains a packet containing one or more banana musa fruits and a container of food for consumption by an animal. The kit may contain additional items such as a device for mixing the banana musa fruits and ingredients or a device for containing the admixture, e.g., a food bowl. In another embodiment, the banana musa fruits are mixed with additional nutritional supplements such as vitamins and minerals that promote good health in an animal.

In another aspect, the present invention provides a means for communicating information about or instructions for one or more of (1) using banana musa fruit to maintain or improve gastrointestinal health, (2) using banana musa fruit to improve stool quality, (3) admixing banana musa fruit with the other components of the present invention, (4) administering banana musa fruit to an animal, alone or in combination with the other elements of the present invention, and (5) using the kits of the present invention for preventing and treating diarrhea or for improving stool quality comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information includes one or more of (1) methods and techniques for combining and administering the banana musa fruits and/or other components and (2) contact information for animals or their caregivers to use if they have a question about the invention and its use. Useful instructions include amounts for mixing and administration amounts and frequency. The communication means is useful for instructing on the benefits of using the present invention and communicating the approved methods for administering the invention to an animal.

In a further aspect, the present invention provides for a use of a composition comprising a gastrointestinal health maintaining or improving amount of at least one banana musa fruit to prepare a medicament. In another, the invention provides for the use of such composition to prepare a medicament for maintaining or improving the gastrointestinal health of an animal. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal. In various embodiments, the medicament further comprises at least one ingredient selected from the group consisting of (1) an ingredient suitable for consumption by an animal, (2) at least one of a gastrointestinal health improving agent selected from the group consisting of probiotics, prebiotics, and anti-diarrhea drugs, and (3) at least one microbial exopolysaccharide.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a banana musa fruit” includes a plurality of such banana musa fruits. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and publications mentioned herein are incorporated herein by reference to the extent allowed by law for the purpose of describing and disclosing the compositions, compounds, methods, and similar information reported therein that might be used with the present invention. However, nothing herein is to be construed as an admission that the invention is not entitled to antedate such disclosure by virtue of prior invention.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

Three different studies were conducted to examine the effect of bananas on helping to control diarrhea in cats with inflammatory bowel disease (IBD) or chronic diarrhea. This is a chronic disease and controlling diarrhea is a life long process. The study design is the same for the 3 studies. The studies, with 11 chronic diarrhea cats, were conducted in a crossover design with 5 cats on a positive control food and 6 cats on test food for 2 weeks. The cats on the control food were then switched to the test food and the cats on the test food were switched to the control food following a 1 week washout period with a commercially available therapeutic food indicated for GI distress. The cats were groomed regularly and The positive control food without banana flakes contains an ingredient, gellan gum, that has been shown to resolve diarrhea (US Patent Application No. 20040180079A1) at a level of 0.3% in the formula (or 1.2% on a dry matter basis) so that the cats were maintained on a food that will help them maintain good stool quality throughout the study. The test foods for the 3 studies were as follows:
a. Study 1: Control food with 0.2% gellan gum plus 0.1% banana flakes. This study was conducted for 12 days.
b. Study 2: Control food without gellan gum but substituted with 0.3% banana flakes (Or 1.2% on a dry matter basis). This study was conducted for 14 days for each period and
c. Study 3: Control food without gellan gum but substituted with 0.3% green banana powder
   Stool quality was monitored for the entire duration of the study. Table 1 shows the composition of the foods (As is) tested in the study.

TABLE 1

| | | Test food | Test food | Test food |
|---|---|---|---|---|
| | Control food | with 0.1% | with 0.3% | with 0.3% |
| | with 0.3% | banana | banana | green banana |
| Analyte | gellan gum | flakes | flakes | powder |
| Protein % | 9.8 | 10.9 | 10.24 | 10.95 |
| Fat % | 5.3 | 6.5 | 8.1 | 6.3 |
| Carbohydrate % | 6.9 | 5.9 | 5.8 | 6.1 |
| Ash % | 1.6 | 1.6 | 1.5 | 1.5 |
| Moisture % | 76.4 | 75.07 | 74.35 | 75.2 |
| Crude fiber % | 0.4 | 0.2 | 0.2 | 0.2 |

Study 1: Test food with 0.1% banana flakes and 0.2% gellan gum. Table 2 shows the average stool quality of the cats when they were on control, washout or test foods. Stool quality was monitored on a scale of 1-5 with 1 being runny and diarrhea stool and 5 being hard and firm stools. Stools considered acceptable and well formed are 4's and 5's. Stools considered acceptable are scored 3's and stools considered unacceptable are scored 1's and 2's. Daily stool quality values were then averaged to get the mean score for the test period.

TABLE 2
Average daily stool quality of cats with chronic
diarrhea on control, washout and test foods

| | Average stool quality ± SD |
|---|---|
| Control food with 0.3% gellan gum | 4.0 ± 0.3 |
| Washout food | 3.7 ± 0.5 |
| Test food with 0.1% banana flakes, | 4.1 + 0.3 |
| 0.2% gellan gum | |

Table 3 shows the frequency of the stool scores over the study period. The data shows that on the control food, there were 71.7% stools scoring 4's and 5's compared to 79.9% when the cats were on the test foods. Both foods did not result in any stools that scored 1's or 2's.

TABLE 3
Frequency distribution of stool scores over the study period for Study 1

| | Stool score | | | | |
|---|---|---|---|---|---|
| | 1 | 2 | 3 | 4 | 5 |
| Control food % | 0 | 0 | 28.4 | 46.3 | 25.4 |
| Test food % | 0 | 0 | 20.1 | 51.5 | 28.4 |

Study 2: Test food with 0.3% banana flakes. Study 2 was conducted similarly to study 1 with the test food containing only 0.3% banana flakes with no gellan gum. The data in Tables 4 and 5 showed again that the control and test foods performed very well to control diarrhea in cats with chronic diarrhea with 91.5% stools scoring 4's and 5's with the control food and 98% with the test food. In fact, it appeared that the test food may be slightly more efficacious than the test food.

TABLE 4
Average stool quality for chronic diarrhea
cats on control, washout and test food.

| | Average stool quality ± SD |
|---|---|
| Control food with 0.3% gellan gum | 4.5 ± 0.2 |
| Washout food | 3.4 ± 0.1 |
| Test food with 0.3% banana flakes | 4.6 ± 0.2 |

TABLE 5
Frequency distribution of stool scores over the study period for Study 2

| | Stool score | | | | |
|---|---|---|---|---|---|
| | 1 | 2 | 3 | 4 | 5 |
| Control food % | 0 | 0 | 8.5 | 35.3 | 56.2 |
| Test food % | 0 | 0 | 2 | 25 | 73 |

Study 3: Test food with 0.3% green banana powder. In study 3 the test food was made with 0.3% green banana powder and no gellan gum. The results are shown in Tables 6 and 7. The data showed that both the control food and test food with 0.3% banana flakes were able to control diarrhea in cats with chronic diarrhea. The stool frequency data showed that 94.1% of the stools scored 4's and 5's on control food and 92.8% of the stools scored 4's and 5's on the test food.

TABLE 6
Average stool quality for chronic diarrhea
cats on control, washout and test food.

| | Average stool quality ± SD |
|---|---|
| Control food with 0.3% gellan gum | 4.5 ± 0.4 |
| Washout food | 3.5 ± 0.2 |
| Test food with 0.3% green banana | 4.5 ± 0.3 |
| powder | |

TABLE 7
Frequency distribution of stool scores over the study period for Study 3.

| | Stool score | | | | |
|---|---|---|---|---|---|
| | 1 | 2 | 3 | 4 | 5 |
| Control food % | 0 | 0 | 5.9 | 30.9 | 63.2 |
| Test food % | 0 | 0 | 7.2 | 35.3 | 57.5 |

In summary, the data shows that the food made with bananas (banana flakes or green banana powder) exerted similar diarrhea-resolving properties as gellan gum, which has been shown to control diarrhea in cats with chronic diarrhea. The two ingredients significantly improved stool quality over the washout food. The data shows that the test food performed as well and sometimes slightly better than the control food (Study 2, Tables 4 and 5).

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for maintaining or improving the gastrointestinal health of an animal susceptible to or suffering from poor gastrointestinal health comprising administering a gastrointestinal health maintaining or improving amount of at least one banana musa fruit to the animal.

2. The method of claim 1 wherein maintaining or improving gastrointestinal health comprises preventing or treating diarrhea.

3. The method of claim 1 wherein improving gastrointestinal health comprises improving stool quality.

4. The method of claim 1 wherein the fruit is a banana.

5. The method of claim 1 wherein the fruit is administered to the animal in amounts of from about 0.1 to about 2 g/kg body weight per day.

6. The method of claim 1 wherein the animal is a canine or a feline.

7. The method of claim 1 further comprising administering the fruit in conjunction with one or more ingredients suitable for consumption by an animal.

8. The method of claim 7 wherein the fruit and ingredients are admixed prior to administration to the animal.

9. The method of claim 7 wherein the ingredient is a food ingredient.

10. The method of claim 7 wherein the fruit comprises from about 0.1% to about 5% of the mixture.

11. The method of claim 1 further comprising administering the fruit in conjunction at least one of a gastrointestinal health improving agent selected from the group consisting of probiotics, prebiotics, and anti-diarrhea drugs.

12. The method of claim 1 further comprising administering the fruit in conjunction with at least one microbial exopolysaccharide.

13. The method of claim 12 wherein the microbial exopolysaccharide is selected from the group consisting of rhamsan, curdlan, xanthan gum, scleroglucan, PS-10 gum, PS-21 gum, PS-53 gum, polysaccharides from Alcaligenes species, PS-7 gum, gellan gum, curdlan, bacterial alginate, dextran, pullulan, baker's yeast glycan, bacterial cellulose, 6-deoxy-hexose-containing polysaccharides, and combinations thereof.

14. The method of claim 12 wherein the microbial exopolysaccharide comprises a gellan gum.

15. The method of claim 12 wherein the microbial exopolysaccharide is administered in amounts of from about 0.05 to about 2 g/kg body weight per day.

16. A composition useful for maintaining or improving the gastrointestinal health of an animal comprising a gastrointestinal health maintaining or improving amount of at least one banana musa fruit and at least one ingredient selected from the group consisting of (1) an ingredient suitable for consumption by an animal, (2) at least one of a gastrointestinal health improving agent selected from the group consisting of probiotics, prebiotics, and anti-diarrhea drugs, and (3) at least one microbial exopolysaccharide.

17. The composition of claim 16 wherein the microbial exopolysaccharide is selected from the group consisting of rhamsan, curdlan, xanthan gum, scleroglucan, PS-10 gum, PS-21 gum, PS-53 gum, polysaccharides from Alcaligenes species, PS-7 gum, gellan gum, curdlan, bacterial alginate, dextran, pullulan, baker's yeast glycan, bacterial cellulose, 6-deoxy-hexose-containing polysaccharides and combinations thereof.

18. The composition of claim 16 wherein the microbial exopolysaccharide comprises a gellan gum.

19. The composition of claim 16 wherein the gastrointestinal health improving agent is an anti-diarrhea drug.

20. A kit suitable for administering banana musa fruits to an animal comprising in separate containers in a single package or in separate containers in a virtual package, as appropriate, at least one banana musa fruit and at least one of (1) a different banana musa fruit, (2) one or more ingredients suitable for consumption by an animal, (3) one or more microbial exopolysaccharides, (4) one or more gastrointestinal tract improving agents selected from the group consisting of probiotics and prebiotics, (5) one or more anti-diarrhea drugs, (6) instructions for how to combine the banana musa fruit and other kit components to produce a composition useful for preventing or treating diarrhea or for improving stool quality, and (7) instructions for how to use the banana musa fruits and other components of the present invention.

21. A means for communicating information about or instructions for one or more of (1) using banana musa fruit to prevent or treat diarrhea, (2) using banana musa fruit to improve stool quality, (3) admixing banana musa fruit with the other components of the present invention, (4) administering banana musa fruit to an animal, alone or in combination with the other elements of the present invention, and (5) using the kits of the present invention for preventing and treating diarrhea or for improving stool quality comprising a document, digital storage media, optical storage media, audio presentation, or visual display containing the information or instructions.

22. The means of claim 21 selected from the group consisting of a displayed web site, brochure, product label, package insert, advertisement, or visual display.

23. A use of a composition that comprises at least one banana musa fruit to prepare a medicament to prepare a medicament for maintaining or improving the gastrointestinal health of an animal.

24. The use of claim 23 wherein the composition further comprises at least one ingredient selected from the group consisting of (1) an ingredient suitable for consumption by an animal, (2) at least one of a gastrointestinal health improving agent selected from the group consisting of probiotics, prebiotics, and anti-diarrhea drugs, and (3) at least one microbial exopolysaccharide.
